(19) United States
(12) Patent Application Publication
(10) Pub. No.: US 2020/0000962 A1
(43) Pub. Date: Jan. 2, 2020

(54) LOAD BEARING CROWDED COLLAGEN CONSTRUCTS

(71) Applicant: Medtronic Vascular, Inc., Santa Rosa, CA (US)

(72) Inventors: Hamed Alavi, Santa Rosa, CA (US); Ya Guo, Santa Rosa, CA (US)

(21) Appl. No.: 16/453,717

(22) Filed: Jun. 26, 2019

Related U.S. Application Data
(60) Provisional application No. 62/693,089, filed on Jul. 2, 2018.

(51) Int. Cl. A61L 27/24; C07K 1/34

(52) U.S. Cl. CPC A61L 27/24; A61L 2430/20; A61L 2430/40; C07K 1/34

(57) ABSTRACT

Load bearing crowded collagen material for use in engineered tissue may be formed by crowding collagen fibrils. The crowding may be performed by dialysis. The fibrils may be formed by neutralizing an acidic solution comprising collagen monomers.

### RELATED APPLICATION

This application claims the benefit of U.S. Provisional Patent Application No. 62/693,089 filed on Jul. 2, 2018, which is hereby incorporated herein by reference in its entirety.

### FIELD

The present disclosure is related to the production and use of load bearing crowded collagen constructs. In particular, the crowded collagen may be used as engineered tissue for implanting in a patient, as a substrate for tissue engineering or drug screening applications, or the like.

### BACKGROUND

Collagen is a self-organizing protein formed from soluble monomers and serves as the main load-bearing component of highly specialized connective tissue membranes such as heart valves. Procollagen as a triple-helical, self-organizing protein may be solubilized into monomers under appropriate conditions, typically acidic conditions, and can self-assemble to form collagen fibrils when brought back to physiological conditions (e.g., buffered saline with pH of 7.4 and 37° C.). However, reports of self-assembly of individual collagen fibrils to form load bearing branched collagen fibrils, supra-fibrils, or collagen fibers similar to what is observed in vivo have been limited. Accordingly, tissue engineers often employ collagen as a degradable cell transport vehicle, rather than as a mechanically strong connective tissue.

U.S. Pat. No. 9,518,106 describes, among other things, the lack of success in engineering connective tissue that can withstand the loads that the tissue may experience in vivo. U.S. Pat. No. 9,518,106 also describes a process in which solutions comprising concentrated collagen monomers are confined within three-dimensional templates and are allowed to polymerize within the template to form collagen fibrils. While higher density collagen material may be formed by the processes described in U.S. Pat. No. 9,518,106, some concerns remain regarding whether the collagen material formed by such processes is only a network of intertwined individual fibrils rather than a branched fibrillar, supra-fibrillar, or fibrous network. In addition, the material formed may not be able to withstand loads that connective tissue may experience in vivo.

### SUMMARY

The present disclosure describes, among other things, a method for forming load bearing crowded collagen constructs. While not intending to be bound by theory, it is believed that the methods described herein are achieved by macromolecular crowding. Macromolecular crowding is a physiologically relevant process that causes an excluded volume effect (EVE), which can induce collagen self-assembly. Unlike previously described methods that concentrate collagen monomers, and then allow the concentrated monomers to polymerize to form collagen fibrils, the present application describes the crowding (volume exclusion) of already polymerized collagen fibrils. By crowding the already formed fibrils, rather than concentrating collagen monomers, the resulting collagen may produce a more consistently strong material containing a branched fibrillar, supra-fibrillar, or fibrous network.

When concentrated collagen monomers are neutralized and polymerized, it is believed that inconsistent fibril formation may result due to reduced mobility of the neutralizing agent once the fibrils begin to form. The ability of the neutralizing agent may be prevented or inhibited from accessing other collagen monomers as the fibrils form from the concentrated monomers, which may result in inconsistent fibril formation. There might also be a significant loss of collagen monomers, fibrils, or monomers and fibrils during such processes as the small monomers or weaker fibrils might not have enough strength to resist against the EVE. In addition, polymerization rates may vary from the surface of the resulting collagen material to the core of the resulting collagen material. For example, the surface may begin to polymerize more quickly than the core due to a relative delay in temperature increase to physiological temperatures at the core due to external heating. Prior disclosures in which concentrated collagen monomers are neutralized and polymerized to form individual collagen fibers do not describe a process or mechanism to cause the individual fibrils to transfer to a network of strong branched fibrils, or to even more mature stages such as a thicker supra-fibrillar network or a fibrous structure. The methods described herein may, among other things, provide for a more uniform, physiologically crowded, and strong collagen material.

The crowded collagen material described herein may be useful for engineering mechanically strong connective tissue. Due to its condensed structure, the collagen material described herein may be used to produce connective tissue having advantages over natural connective tissue or previously engineered tissues. For example, engineered tissue comprising the crowded collagen described herein may be one or more of: more durable; less prone to calcification; less prone to thrombus formation; less likely to elicit an immune response; and capable of tuning the structure prior to fabrication, relative to previously engineered connective tissues. The load bearing crowded collagenous tissues may be more durable than native and previous prosthetic tissues due to one or more of their condensed and crowded collagen structure and stronger bonds between the individual fibrils/supra-fibrils/fibers. Because of the formation of a crowded, extra-packed and branched network of fibrils/fibers, the surface of the collagen constructs may resist calcium deposition and diffusion (extrinsic and intrinsic calcification) and may reduce cell (live or dead)-induced intrinsic and extrinsic calcification. Due to the smooth surface of these constructs, they may cause less damage to red blood cells and thrombus formation. In addition, these constructs may be less susceptible to elicit an immune reaction because their crowded structure can block macrophage penetration caused by monocyte differentiation. Blocking macrophage penetration would reduce inflammatory-induced calcification, fibrous capsule formation, and therefore intrinsic, extrinsic, or intrinsic and extrinsic calcification.

In some embodiments disclosed herein, a method includes forming collagen fibrils and crowding the collagen fibrils to form collagen constructs having a collagen density of 100 mg/ml or greater. For example, the collagen constructs may have a collagen density of about 250 mg/ml or greater or about 700 mg/ml or greater. In some embodiments, the collagen construct has a collagen density of from about 700 mg/ml to about 1000 mg/ml. The fibrils may be crowded by dialysis.

Preferably, the fibrils are formed from a solution of collagen monomers at a concentration of about 50 mg/ml or less. For example, the fibrils may be formed from a solution of collagen monomers at a concentration of about 40 mg/ml or less, about 35 mg/ml or less, about 30 mg/ml or less, about 25 mg/ml or less, about 20 mg/ml or less, about 15 mg/ml or less, about 10 mg/ml or less, or about 5 mg/ml or less. The fibrils may be formed by neutralizing the pH of the solution (and lowering down the concentration) followed by polymerization. By forming the fibrils in solutions comprising such relatively low concentrations of collagen monomers, the neutralizing agent should be able to more uniformly affect all the monomers and produce more uniform fibril formation.

Once formed, the fibrils may be crowded through, for example, dialysis to form one or more of a branched network of fibrils, supra-fibrils, or fibers. In some embodiments, pre-incubation may occur prior to polymerization stage to further enhance self-assembly and to allow the production of thicker supra-fibrils, a network of fibers, or thicker supra-fibrils and a network of fibers.

In some embodiments, the crowding, such as via dialysis, may be combined with application of a load to produce a strong network of thick supra-fibrils or fibers. Any suitable load may be applied to the construct during crowding. For example, one or more of shear, bending, and normal (tensile/compression) stresses may be applied to the constructs. Crowding may be performed in more than one stage. For example, crowding may be performed prior to polymerization (one-stage incubation) and after polymerization. Collagen monomers may be combined with proteins such as but not limited to fibronectin and laminin to further enhance the self-assembly process and to produce hybrid constructs.

In some embodiments disclosed herein, an engineered tissue comprises a collagen construct comprising a plurality of collagen fibrils, supra-fibrils, or fibers having a density of about 100 mg/ml or greater. For example, the constructs containing a plurality of collagen fibrils, supra-fibrils, or fibers may have a density of about 250 mg/ml or greater or may have a density from about 700 mg/ml to about 1000 mg/ml. The plurality of collagen fibrils, supra-fibrils, or fibers in the constructs may be cross-linked to further enhance the strength and longevity of the constructs and to avoid in vivo degradation. These constructs might be also used “as is” as substrates/scaffolds for traditional in vitro, in situ, or off-the-shelf tissue engineering applications. With in vitro tissue engineering, the cells may be cultured on the substrate prior to implantation. With in situ tissue engineering applications, the scaffold may be implanted cell-free to be populated by cells inside the body. In off the shelf tissue engineering applications, the cells may be cultured on the scaffold in vitro to form a matrix, the scaffold may then be decellularized prior to implantation, and the body may repopulate the scaffold once implanted. The collagen constructs in their original form may be substantially cell-free, or free of cells.

The collagen constructs, such as engineered tissue, may be used for any suitable purpose. In some embodiments, the engineered tissue is used to form a whole heart valve (e.g. including all or a combination of the lumen, the leaflets, and the skirts), valve leaflets, valve skirts, valve lumen, or a portion thereof, for a prosthetic heart valve. In some embodiments, the engineered tissue is used to form a paravalvular wrap to prevent leakage following implantation of a prosthetic heart valve. In some embodiments, the engineered tissue is used to form blood vessels or arterial grafts. In some embodiments, the engineered tissue is used to form cardiac patches. In some embodiments, the engineered tissue is used to form skin patches. In some embodiments, the engineered tissue is used for wound healing or tissue repair applications. In some embodiments, the engineered tissue is used for facial reconstructive applications such as but not limited to ear and nose reconstruction.

### BRIEF DESCRIPTION OF THE DRAWINGS

FIG. 1 is a schematic drawing illustrating stages in a process of collagen fiber formation.

FIGS. 2-5 are flow diagrams illustrating embodiments of methods or aspects of methods described herein.

FIGS. 6A-C are schematic views of an engineered prosthetic heart valve comprising a collagen construct according to embodiments described herein. FIG. 6A is a top plan view; FIG. 6B is a cut-away perspective view; and FIG. 6C is a perspective view.

FIG. 7 is a schematic perspective view of an engineered paravalvular wrap comprising a collagen construct according to embodiments described herein.

FIG. 8 is a stress-strain plot of various tested collagen material and tissue.

FIGS. 9A-D are plots of Extension Modulus (9A), Elastic Modulus (9B), Max or Ultimate Tensile Stress (9C), and Strain at Max Stress (9D) of various tested collagen material and tissue.

FIG. 10 is a graph of resistance to enzymatic (pronase) degradation of porcine collagen tissue, bovine collagen tissue, and collagen material made in accordance with the teachings presented herein.

FIGS. 11A-D are a number of microscopy images of collagen material and collagen tissue: 11A (150 mg/ml collagen material, 6000× magnification); 11B (Evolut Pro collagen tissue, 3000× magnification); 11C (150 mg/ml collagen material, rinsed, 6000× magnification); and 11D (250 mg/ml collagen material, rinsed, 6000× magnification).

FIGS. 12A-B are microscopy images of collagen material preincubated four hours at 4° C. following neutralization: 12A (not rinsed, 6000× magnification); and 12B (rinsed, 6000× magnification).

FIGS. 13A-B are microscopy images of collagen material produced with (13A, 10× magnification) and without (13B, 10× magnification) bi-directional flow during crowding.

### DETAILED DESCRIPTION

The following detailed description is illustrative in nature and is not intended to limit the scope, applicability, or configuration of inventive embodiments disclosed herein in any way. Rather, the following description provides practical examples, and those skilled in the art will recognize that some of the examples may have suitable alternatives. Embodiments will hereinafter be described in conjunction with the appended drawings, which are not to scale (unless so stated), wherein like numerals/letters denote like elements. However, it will be understood that the use of a number to refer to a component in a given drawing is not intended to limit the component in another drawing labeled with the same number. In addition, the use of different numbers to refer to components in different drawings is not intended to indicate that the different numbered components cannot be the same or similar to other numbered components. Examples of constructions, materials, dimensions and fabrication processes are provided for select elements and all other elements employ that which is known by those skilled in the art.

Reference will now be made in greater detail to various embodiments of the subject matter of the present disclosure, some embodiments of which are illustrated in the accompanying drawings.

The present disclosure describes, among other things, a method for forming load bearing crowded collagen constructs, and articles, such as engineered tissue, comprising the crowded collagen constructs. The method for forming crowded collagen constructs comprises forming collagen fibrils from collagen monomers and crowding (volume exclusion) the formed collagen fibrils to form the load bearing crowded collagen constructs. The method may optionally include one or both of confinement (strain stabilization) and stress-induced self-assembly of the collagen fibrils.

As used herein, “collagen” is a protein component of an extracellular matrix having a tertiary structure that includes polypeptide chains intertwining to form a triple helix or having a characteristic amino acid composition comprising GLY-X-Y repeat units, or a fragment thereof. X may be proline, and Y may be hydroxyproline. A collagen may be any collagen known in the art, such as one of collagen Type 1-29. Preferably, the collagen is a fibrillar collagen such as Types I, II, III, V and XI, which serve as a principal structural component in load-bearing extracellular matrix (ECM).

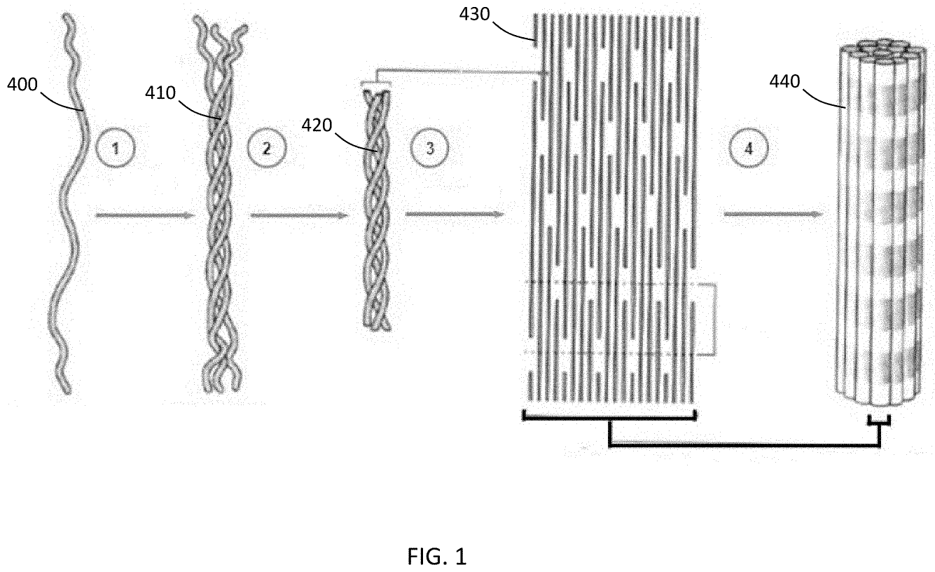

As used herein, a “collagen fibril” is an assembly of collagen molecules having a diameter in a range from about 10 nm to about 500 nm. A plurality of fibrils may intertwine to create a structure having greater strength than a structure consisting of non-intertwined fibrils. In some embodiments described herein, the collagen may comprise a network of branched collagen fibrils. The “network of branched collagen fibrils,” may be observed via microscopy. In some embodiments described herein, collagen constructs comprise collagen supra-fibrils. As used herein, “collagen supra-fibrils” are collagen fibrils that are locally organized and oriented. In some embodiments, the collagen constructs comprise collagen fibers. As used herein, a “collagen fiber” is an assembly of multiple collagen fibrils into a fiber structure having a diameter in a range from about 1 micrometer to about 500 micrometers. Various stages of collagen fiber formation are depicted in schematic form in FIG. 1.

As shown in FIG. 1, precursor α chains 400 assemble to form a procollagen triple helix with loose ends 410 in step 1. In step 2, procollagen peptidase cleaves the procollagen triple helix with loose ends 410 to produce a collagen molecule 420. In step 3, collagen molecules 420 are assembled to produce a collagen fibril 430 having a diameter of from about 10 nm to about 500 nm. In step 4, the smaller collagen fibrils 430 assemble to form larger diameter collagen fibers 440 having a diameter of about 1 micrometer to about 500 micrometers.

Any known collagen may be used in the methods described herein. The collagen may be isolated or derived from a natural source or manufactured in any suitable manner. For example, the collagen may be biochemically or synthetically manufactured, produced through genetic engineering, or the like. Collagen may also be purchased from any one of a number of commercial vendors.

Collagen may be obtained from any suitable mammalian tissue. For example, collagen may be obtained from tendons, bones, cartilage, skin, or any other suitable organ. In some embodiments, collagen is obtained from rat tail tendon, porcine or calf skin.

Regardless of the source, the collagen may be purified. The purified collagen may be in any suitable form, such as a powder. Powdered collagen is commercially from, for example, Sigma-Aldrich and Thermo Fisher.

Purified collagen may be reconstituted in a suitable solution. The solution is preferably acidic. More preferably, the solution is an aqueous solution having a pH of about 4 or less, such as from about 2 to about 3.5. In such acidic solutions, the reconstituted collagen comprises collagen monomers. Preferably, the solution comprises a sufficient amount of acetic acid or hydrochloric acid to achieve a pH of about 2 to about 3.5. Reconstituted collagen is commercially available from, for example, Sigma-Aldrich.

The reconstituted acidic collagen solution may be neutralized to cause formation of collagen fibrils from the collagen monomers. As used herein, “collagen fibrils” means an association of several collagen monomers into a structure that appears fibrous with suitable magnification with thickness of fibrils usually between 10 nm to 500 nm. The fibrils may self-assemble to form supra-fibrils, fibers, or supra-fibrils and fibers. Collagen fibers typically have thickness in a range from about 1 micrometer to about 500 micrometers.

The reconstituted collagen solution may be neutralized in any suitable manner. For example, the reconstituted collagen solution may be neutralized by adding a base to the solution. Any suitable base may be used. For example, the base may be sodium hydroxide. Preferably, the solution is neutralized by adjusting the pH of the solution to a pH of 5 or greater. For example, the solution may be adjusted to a pH of about 5 to about 10, such as about 5.5 to about 9.5, about 6 to about 9, about 6.5 to about 8.5, or about 6.5 to about 8. Neutralizing the solution allows for fibril formation.

The neutralized solution may also be altered in any other suitable manner. For example, a suitable buffer, such as phosphate buffered saline or the like, may be added to the solution. By modifying the solution to more closely mimic in vivo fluid, fibril formation may be encouraged. The neutralized solution may be added or injected to a confined space, such as a cassette, having a shape of a desired tissue to be formed by the collagen. The confined space might have adjustable dimensions.

The neutralized solution may be heated to facilitate formation of collagen fibrils from the collagen monomers. In some embodiments, the neutralized solution is heated to about 37° C. In some embodiments, the neutralized solution is kept at a temperature below 37° C. prior to heating the solution. The rate of fibril formation may be lower at lower temperatures than at higher temperatures, such as 37° C. Without intending to be bound by theory, it is believed that a slower rate of fibril formation may provide for more uniform fibril formation and more complete fibril formation.

In some embodiments, the neutralized solution is retained at about 4° C. for a period of time to permit slow fibril formation. For example, the neutralized solution may be retained at 4° C. for about 4 hours to about 48 hours, such as about 8 hours to about 36 hours, or about 24 hours. After retaining the neutralized solution at about 4° C., the neutralized solution may optionally be incubated at 37° C. for any suitable time to complete polymerization and fibril formation. For example, the neutralized solution may be incubated at 37° C. for about 15 minutes to about 4 hours, such as from about 30 minutes to about 2 hours, or for about 1 hour.

In some embodiments, the neutralization is performed using pH-controlled dialysis. Controlling the pH of the dialysis agent can be performed manually such as by titration or automatically such as using a pH controller. pH-controlled dialysis can be performed at low temperature such as 4° C. or higher temperature such as 37° C. or in sequences. Dialysis during such stage might or might not induce molecular crowding.

The neutralized solution may comprise any suitable concentration of collagen. Preferably, the neutralized solution comprises collagen at a concentration of about 50 mg/m or less, such as about 30 mg/ml or less, about 25 mg/ml or less, about 20 mg/ml or less, about 15 mg/ml or less, or about 10 mg/ml or less. By forming the fibrils in solutions comprising such concentrations of collagen monomers, more uniform and complete consumption of the monomers to form fibrils may result compared to neutralized solutions having higher concentrations of collagen monomers. In some embodiments, the collagen monomer solution can be initially combined with other molecules such as but not limited to polyethylene glycol or glycosaminoglycans such as hyaluronan acid and then neutralized to achieve bi- or tri-molecular crowding.

The fibrils may then be subjected to dialysis to concentrate and crowd the fibrils. Dialysis may be performed in a confined space, such as a cassette, having a shape of a desired tissue to be formed by the collagen. The fibrils may self-assemble to form supra-fibrils, fibers, or supra-fibrils and fibers, prior to, during, or after the dialysis.

The dialysis may be performed under any suitable conditions. For example, the duration and temperature of the dialysis may be suitably varied. Preferably, the dialysis is performed at a temperature of about 4° C. to about 37° C. The duration of the dialysis, which may include crowding the solution against which the collagen is dialyzed, is preferably from about 30 minutes to 48 hours, such as from about 2 hours to about 36 hours, from about 12 hours to about 30 hours, or about 24 hours. Any suitable membrane may be used to perform the dialysis. For example, the membrane may have a molecular weight cut off in range from about 1000 Da to about 20,000 Da, such as from about 2000 Da to about 10,000 Da, or about 7000 Da. The collagen may be dialyzed against any suitable solution. For example, the collagen may be dialyzed against polyethylene glycol (PEG), Dextran, a water-soluble polymer, Glycosaminoglycans, Hyaluronan Acid, or any other suitable solution that contains large molecules. Preferably, the collagen is dialyzed against PEG. The PEG may have any suitable molecular weight. For example, the PEG may have a number average molecular weight (Mn) from about 2000 Da to about 30,000 Da, such as from about 4000 Da to about 25,000, or from about 8000 Da to about 20,000 Da. Additionally, the dialysis solution may have any suitable concentration. For example, from about 10% (w/v) to about 50% (w/v) in water or phosphate buffered saline (PBS), or from about 20% (w/v) to about 40% (w/v).

Preferably, the molecular weight cut off (MWCO) of the dialysis membrane is lower than the molecular weight (MW) of a molecule, such as PEG, in the solution against which the collagen is dialyzed. If MWCO is higher than MW of PEG overcrowding may happen.

Preferably, the dialysis results in a collagen concentration of 100 mg/ml or greater, such as 250 mg/ml or greater, or 700 mg/ml or greater. In some embodiments, the collagen concentration is from about 700 mg/ml to about 1000 mg/ml. The collagen density can be measured by any proper biochemical or biophysical method such as any suitable spectrophotometry or calorimetry methods. Additionally, collagen density can be measured by calculating the total volume of the construct and its ratio to the initial volume.

The crowded collagen material described herein may be useful for engineering mechanically strong connective tissue. Due to its crowded structure, the collagen material described herein may be used to produce connective tissue having advantages to natural connective tissue or previously engineered tissues. For example, engineered tissue comprising the crowded collagen described herein may be one or more of: more durable; less prone to calcification; less prone to thrombus formation; less likely to elicit an immune response; and capable of tuning the structure prior to fabrication, relative to previously engineered connective tissues. The load bearing crowded collagenous tissues may be more durable than native and previous prosthetic tissues due to their condensed and crowded collagen structure, stronger bonds between the individual fibrils/supra-fibrils/fibers, or condensed and crowded structure and stronger bonds. Because of the formation of a crowded, extra-packed and branched network of fibrils/fibers, the surface of the collagen construct may resist calcium deposition and diffusion (extrinsic and intrinsic calcification) and may reduce cell (live or dead)-induced intrinsic and extrinsic calcification. Due to the smooth surface of these constructs due to coverage with PEG, they may cause less damage to red blood cells and reduce thrombus formation due to collagen-blood interaction. In addition, the constructs may be less susceptible to elicit an immune reaction because their crowded structure can block macrophage penetration caused by monocyte differentiation. Blocking macrophage penetration may reduce inflammatory-induced calcification, fibrous capsule formation, and therefore intrinsic and/or extrinsic calcification.

In some embodiments, dialysis is performed with dynamic flow of the solution against with the collagen is dialyzed. The jet stream from fluid flow produces a shear force that can affect alignment and orientation of the collagen fibrils and accelerate the self-assembly process to form thicker and elongated fibrils, supra-fibrils, and fibers. The flow can be produced with any suitable methods. In some embodiments, the flow is produced using one or more of pumps submerged in the dialysis solution or connected to the dialysis solution container with the jet stream passing over the surface of flat constructs. In some embodiments, the flow is generated using a flow simulator system when the constructs have complex shapes, such as a heart valve shape. The rate of flow of the fluid in circulation may be varied to simulate high and low shear rates. The flow rate may be adjusted by the spatial location of the pumps relative to the cassette. The direction of the circulation may be varied by orientating the pumps and the direction of their jet streams. In some embodiments, one submersible pump may be used to produce a flow parallel to the surface of the flat collagen sheets. The pump may be placed in a way that the flow passes over only one surface (top or bottom) or both surfaces. In some embodiments, two submersible pumps may be used in an angle with respect to each other to produce bi-directional flow streams. The flow streams may be perpendicular to each other. In some embodiments, one of the pumps may be placed in a way to produce a jet stream passing over a top surface of a collagen sheet and the flow from another pump passing over a bottom surface of the collagen sheet. These flow streams may be angled relative to each other, such as 90 degrees to one another. This way the collagen fibrils/fibers in one side of the sheet have a direction perpendicular (or with an angle) to collagen fibers in the other side of the sheet. This may produce a collagen construct with anisotropic behavior. Dynamic flow (shear force) may be applied at any stage during the crowded collagen process such as reconstitution, neutralization, pre-incubation, one-stage incubation, polymerization, dialysis, crosslinking, or a combination thereof.

Fiber or fibril alignment or orientation, such as described above, may serve to enhance long term durability and mechanical performance of the constructs as the case in native collagenous tissues where the collagen fibers are aligned in certain directions. In addition, applying the shear load during dialysis may expedite and enhance the self-assembly process. In case of the load bearing tissues, such as heart valve leaflets, the collagen and elastin fibers may be aligned differently in their own sub-layers to provide anisotropic characteristics. Another way to produce aligned collagen fibrils/fibers would be application of bending stress/strains or application of normal (tensile or compression) stress/strains. In some embodiments, the collagen constructs may be pulled by hanging weights or placed in tension boards to induce directionality. As with the dynamic flow (shear force), other load regimes or a combination of such loading may be applied at any stage during crowded collagen process such as reconstitution, neutralization, pre-incubation, one-stage incubation, polymerization, dialysis, crosslinking, or a combination thereof.

The crowded collagen material can be used in combination with other materials such as but not limited to fabrics, polymers, biologic materials, and metals to form hybrid structures. These materials can provide additional features to improve the chemical, mechanical, or biological performance of the final hybrid structure. They can further reinforce the constructs or improve the biocompatibility and blood-compatibility. The added materials can be in any suitable forms such as solutions, gels, solid particles, woven or non-woven materials or a combination thereof. They can be distributed evenly throughout the crowded collagen, only be applied to specific sections in an uneven distribution or be applied as surface coating. In some embodiments, the added material can be elastin. Elastin can be neutralized with collagen in any ratios such as collagen to elastin ratios from about 20:1 to about 1:20; or about 10:1 to about 1:1; or about 5:1 to about 2:1. In some embodiments, the added materials can be fibronectin or laminin. In some embodiments, the added materials can be used to strengthen certain parts of the constructs. For example, in the form of a heart valve leaflet the basal part of the leaflets where they are sewn to the fabric or are attached to the frame can be strengthened. Any structural tuning processes described in this document can be applied to the hybrid structures, as well.

Following crowding and concentration, the resulting collagen material may be optionally cross-linked with a suitable cross-linking agent. For example, the resulting collagen material may be cross-linked with one or more of glutaraldehyde, a diisocyanate, a polyepoxy compound, gamma irradiation, ultraviolet irradiation, transglutaminase, an ether, an epoxide, a carbodiimide, a natural crosslinker such as genipin, or the like, or a combination thereof. Some examples of cross-linking agents and processes that may be employed include glutaraldehyde, hexamethylene diisocyanate (HMDI), 1-ethyl-3-(3-dimethylaminopropyl) carboiimide (EDC), rose bengal, riboflavin, ribose, glucose, genipin, oleuropein, transglutaminase, and the like.

Cross-linking the fibers or fibrils may serve to provide one or more advantageous properties to the resulting collagen material. For example, cross-linking may enhance the strength of the resulting collagen material, may result in tissue that is less susceptible to cell or calcium infiltration, may result in tissue that is less susceptible to degradation, and the like. In some embodiments, the resulting collagen material is not crosslinked. The collagen material may be used “as is” for in vitro, in situ, or off-the-shelf tissue engineering. In some embodiments, the collagen material is used for drug screening application.

The structure, concentration, and fiber orientation of the resulting collagen material, which may be used as or for producing engineered tissue may be adjusted by adjusting parameters associated with forming the collagen material. For example, the time and temperature of fibril formation from collagen monomers may be adjusted as appropriate. The pH, osmolality, and other solution properties of the neutralized solution for forming the collagen fibrils or fibers may be appropriately adjusted. The duration of dialysis to concentration the fibril or fibrils, the temperature of the dialysis, the molecular weight cut off of the dialysis membrane, the solution against with the collagen is dialyzes, and the like may be adjusted as appropriate. Fiber orientation, hybrid structure, or cross-linking may or may not be performed as appropriate. These and other parameter may be adjusted to produce engineered collagen tissue having desired properties.

The collagen material may have any suitable mechanical properties. Preferably, the collagen material has an elastic modulus of about 20 MPa or greater. The elastic modulus may be measured when uni-axially stretching a coupon of collagen material of 40 mm×10 mm at a rate of 10 mm/min and ending when the percent peak load drops 40%. Preferably, the collagen material has a tensile modulus of about 40 MPa or greater, about 60 MPa or greater, about 80 MPa or greater, or about 100 MPa or greater.

Preferably, the collagen material has an extension modulus of about 1 MPa or greater, such as about 10 MPa or greater. In some embodiments, the collagen material may have an extension modulus of about 25 MPa or greater of about 50 MPa or greater. Extension modulus is the slope of the tangent line to the stress-strain curve of soft tissue (viscoelastic) materials at the low or physiological strains. Elastic modulus is calculated similar to extension modulus; however, it belongs to higher strain amounts where the stress-strain curve is at the elastic section.

The crowded collagen material may have any suitable thickness. In some embodiments, the collagen material has a thickness of from about 0.05 mm to about 1 mm, such as from about 0.1 mm to about 0.4 mm. The thickness of the collagen material may be adjusted depending on the intended use of the collagen material.

Collagen material produced by the methods described herein may be used to engineer any suitable tissue. For example, the collagen material may be used to engineer soft tissue structures, cartilaginous structure, connective tissue, vascular tissue, bone tissue, and the like. The collagen material may be used to engineer soft tissue of the trachea, epiglottis, vocal cords, and the like. The collagen material may be used to engineer articular cartilage, nasal cartilage, tarsal plates, tracheal rings, thyroid cartilage, arytenoid cartilage. The collagen material may be used to engineer vascular grafts and components thereof. The collagen material may be used to engineer sheets for topical applications or for repair of organs such as livers, kidneys, and pancreas. The collagen material may be used to engineer bone, dental structures, joints, cartilage, skeletal muscle, smooth muscle, cardiac muscle, tendons, menisci, ligaments, blood vessels, stents, heart valves, corneas, ear drums, nerve guides, tissue patches or sealants, a filler for missing tissues, skin, or the like.

In some embodiments, the collagen is produced having a predetermined shape defined by, for example, the shape of a cassette or confined container used for dialysis. For examples, the cassette may cause formation of a collagen tissue in the shape of a whole heart valve with tubular lumen and continuously attached leaflets (without the need to suture the leaflets), a total heart valve without the lumen, a heart valve leaflet, a heart valve skirt, a heart valve frame, a paravalvular wrap, or a blood vessel.

In some embodiments, the collagen may be shaped by manual processing, such as suturing, sealing, stapling, cutting, or the like. In case of a heart valve, the formed valve may or may not be pressure fixed.

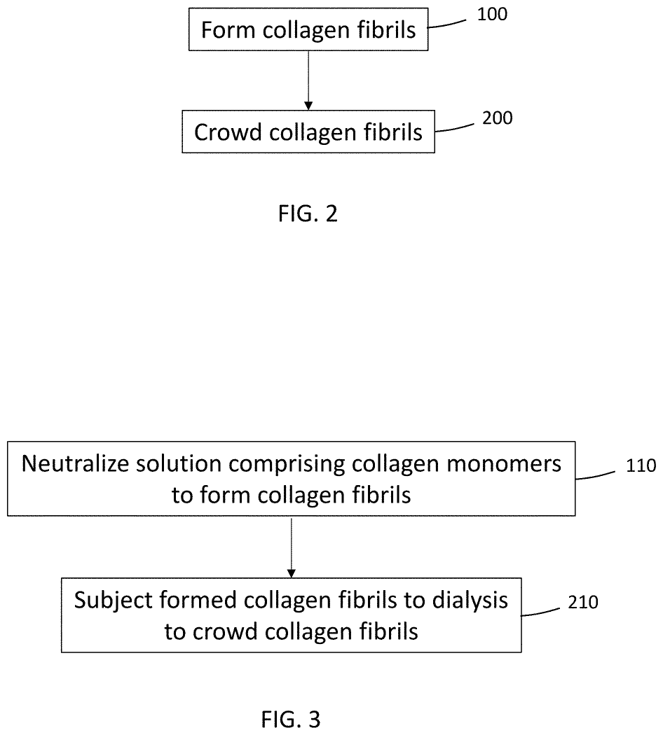

Referring now to FIGS. 2-7, illustrative methods and articles are depicted.

FIG. 2 illustrates a method in which collagen fibrils are formed (100) and the formed collagen fibrils are crowded (200). The collagen fibrils may be crowded under conditions sufficient to cause the collagen to have a density of 100 mg/ml or greater, such as 250 mg/ml or greater, 700 mg/ml or greater, or from 700 mg/ml to 1000 mg/ml. Preferably, the collagen fibrils are crowded under conditions that cause the fibrils to form intertwined fibrils, a network of branched fibrils, supra-fibrils, or fibers.

The formation of intertwined fibrils, a network of branched fibrils, supra-fibrils, or fibers may be caused or controlled in any suitable manner. For example, a load may be applied to the collagen construct as the fibrils are being crowded. Any suitable load may be applied to the construct during crowding. For example, one or more of shear, bending, and normal (tensile/compression) stresses may be applied to the constructs.

One or more additional components may be included while the collagen is being crowded to form a hybrid construct. Examples of suitable additional components include a fabric, a polymer, a biologic material, and a metal. In some embodiments, the additional component comprises elastin. In some embodiments, the additional component comprises fibronectin or laminin.

The collagen fibrils may be cross-linked at any suitable time and in any suitable manner. In some embodiments, the collagen fibrils are not cross linked.

FIG. 3 illustrates a method in which collagen fibrils are formed by neutralizing a solution comprising collagen monomers (110) and collagen fibers are crowded by subjecting the fibrils to dialysis (210). The solution comprising the collagen monomers may be neutralized in any suitable manner. For example, the solution may be acidic and may be neutralized by adding a base. Preferably, the solution is neutralized to a pH in a range from about 5 to about 10 to form the collagen fibrils. The solution may comprise any suitable concentration of collagen monomers. For example, the solution may comprise collagen monomers at a concentration of 50 mg/ml or less, such as 30 mg/ml or less, 25 mg/ml or less, 20 mg. ml or less, 15 mg/ml or less, or 10 mg/ml or less.

Any suitable dialysis protocol may be formed to crowd the collagen fibrils. Preferably, the fibrils are dialyzed against a solution comprising polyethylene glycol (PEG). The PEG may have any suitable molecular weight. For example, the PEG may have a number average molecular weight (Mn) in a range from about 2000 to about 30,000. Preferably, the dialysis is performed using a membrane having a molecular weight cut off in a range from about 1000 to about 20,000.

FIG. 4 illustrates a method in which collagen fibrils are formed by neutralizing a solution comprising collagen monomers (113), the neutralized solution is preincubated at 4° C. (115), and then the neutralized solution is incubated at a temperature greater than 4° C. (117). For example, the temperature of the incubation (117) may be about 37° C. The incubation (117) may be for any suitable time, such as from about 15 minutes to about 4 hours. The preincubation at 4° C. may be for any suitable time, such as from about 4 hours to about 48 hours.

FIG. 5 illustrates a method in which collagen fibrils are concentrated in a cassette (213), and a collagen construct is removed from the cassette (215). As used herein, a “cassette” is any suitable three-dimensional structure in which the collagen fibrils may be crowded. Preferably, the cassette has an internal shape that corresponds to the desired shape of the collagen construct. For example, if the collagen construct is to be used as a prosthetic heart valve, the internal shape and dimensions of the cassette is the shape and dimensions of the prosthetic heart valve.

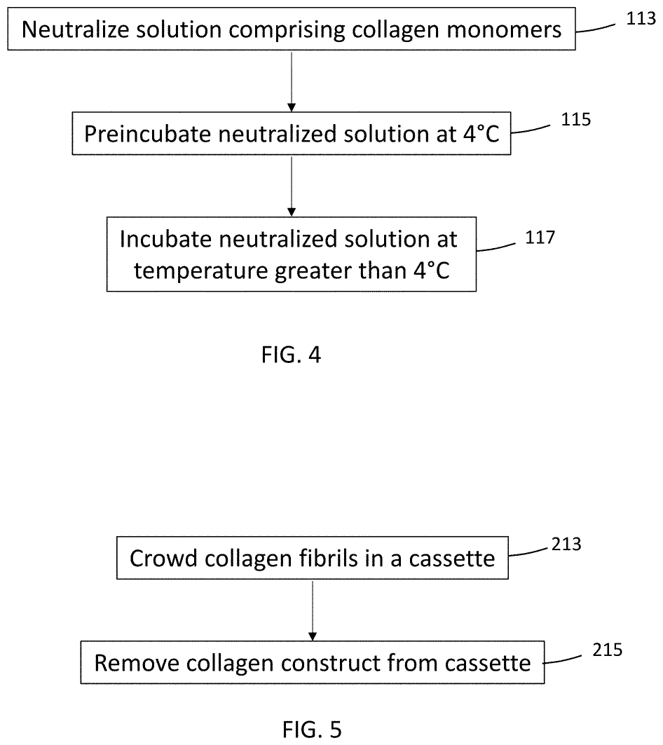

FIGS. 6A, 6B, and 6C illustrate an engineered prosthetic heart valve comprising a collagen construct. In particular, FIG. 6A shows a top view of a closed valve with three valve sinuses, FIG. 6B shows a perspective sectional view of the closed valve, and FIG. 6C shows an exterior view.

One consideration in the design of a prosthetic heart valve is the architecture of the valve sinus. Valve sinuses 12 are dilations of the vessel wall that surround the natural valve leaflets. Typically, in the aortic valve, each natural valve leaflet has a separate sinus bulge 12 or cavity that allows for maximal opening of the leaflet at peak flow without permitting contact between the leaflet and the vessel wall. As illustrated in FIGS. 6A, 6B, and 6C, the extent of the sinus 12 is generally defined by the commissures 11, wall of the prosthetic heart valve 13, inflow end 14, and outflow end 15. The proximal intersection between the sinus cavities define the commissures 11.

FIGS. 6B and 6C also show the narrowing diameter of the sinuses at both inflow end 14 and outflow end 15, thus forming the inflow and outflow annuli of the sinus region. Thus, the valve sinuses form a natural compartment to support the operation of the valve by preventing contact between the leaflets and the vessel wall, which, in turn, may lead to adherence of the leaflets and/or result in detrimental wear and tear of the leaflets. The valve sinuses are also designed to share the stress conditions imposed on the valve leaflets during closure when fluid pressure on the closed leaflets is greatest. The valve sinuses further create favorable fluid dynamics through currents that soften an otherwise abrupt closure of the leaflets under conditions of high backflow pressure. Lastly, the sinuses ensure constant flow to any vessels located within the sinus cavities.

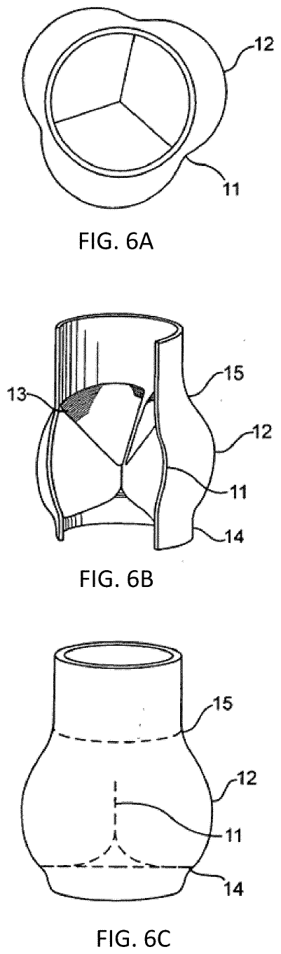

FIG. 7 illustrates a paravalvular wrap 300 comprising a collagen construct as described herein. The wrap 300 comprises a wall 310 defining a lumen 135 through the length of the wall. The wrap 300 may be placed around a vessel of a patient where leaking occurs or may occur. For example, the wrap 300 may be placed around a vessel in which a prosthetic valve, such as the valve depicted in and described regarding FIGS. 6A-C.

As used in this specification and the appended claims, the singular forms “a”, “an”, and “the” encompass embodiments having plural referents, unless the content clearly dictates otherwise.

As used in this specification and the appended claims, the term “or” is generally employed in its sense including “and/or” unless the content clearly dictates otherwise. The term “and/or” means one or all of the listed elements or a combination of any two or more of the listed elements. The use of a slash “I” between words means “and/or.”

As used herein, “have”, “having”, “include”, “including”, “comprise”, “comprising” or the like are used in their open-ended sense, and generally mean “including, but not limited to”. It will be understood that “consisting essentially of”, “consisting of”, and the like are subsumed in “comprising” and the like. As used herein, “consisting essentially of,” as it relates to an article, means that the components of the article are limited to the enumerated components and any other components that do not materially affect the basic and novel characteristic(s) of the article.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Methods for manufacturing load bearing crowded collagen material and resulting crowded collagen material are illustrated in the following examples. The examples are provided for illustrative, non-limiting purposes.

### Examples

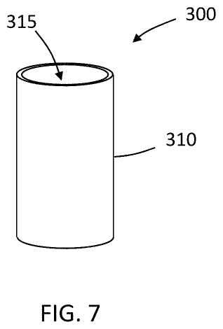

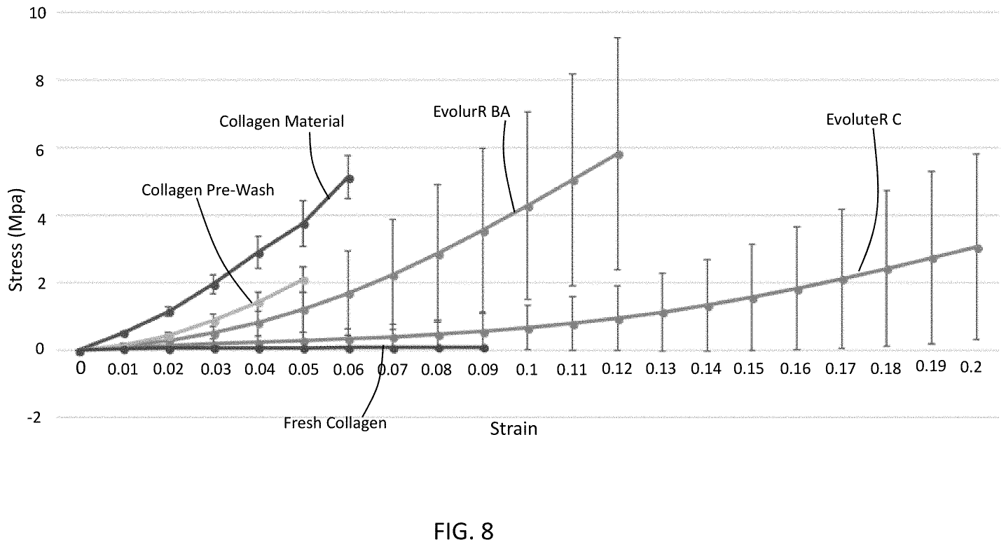

Collagen material was prepared from an acid soluble 10.4 mg/ml collagen solution. The solution was neutralized and polymerized at 1 hour with NaOH (0.1M) in 10× phosphate buffered saline (PBS) at 37° C., which was then dialyzed overnight against PEG at 37 C and crosslinked in 0.2% glutaraldehyde for 1 hour. Rectangular coupons of dimensions 40 mm×10 mm were cut, and the modulus of the coupons was tested by stretching at a rate of 10 mm/min and ending when the percent peak load dropped 40%. The mechanical properties of tissue were derived from commercially available prosthetic heart valves (Medtronic, Inc.'s Evolut™ transcatheter aortic valve replacement system) in base-to-apex direction aka “EvolutR BA” and circumferential direction aka “EvolutR C”. A resulting stress-strain curve is shown in FIG. 8 and graphs of extension Modulus, Elastic Modulus, Max or Ultimate Tensile Stress, and Strain at Max Stress are shown in FIGS. 9A-D. Results are shown for the collagen material, pre-washed collagen, and fresh collagen, as wells as for the EvolutR BA and EvolutR C replacement valves. “Collagen Material” is the collagen that was prepared the same way described above. “Collagen pre-washed” was prepared the same way as collagen material; however, the sheets were soaked in 1×PBS for 30 mins prior to crosslinking in 0.2% glutaraldehyde. Finally, fresh collagen is the collagen material but with no crosslinking. In general, the collagen material had a modulus greater than tissue from the commercially available replacement valves.

Differential Scanning calorimetry (DSC) was used to determine shrink (denaturation) temperature of the collagen material, which was 83.73° C. Shrink temperature above 80° C. is comparable to fixed bovine and fixed porcine tissues used in commercial valves and denotes efficient crosslinking of the material with 0.2% glutaraldehyde. Volumetric methods were used to determine collagen concentration of the collagen material, which was about 150 mg/ml.

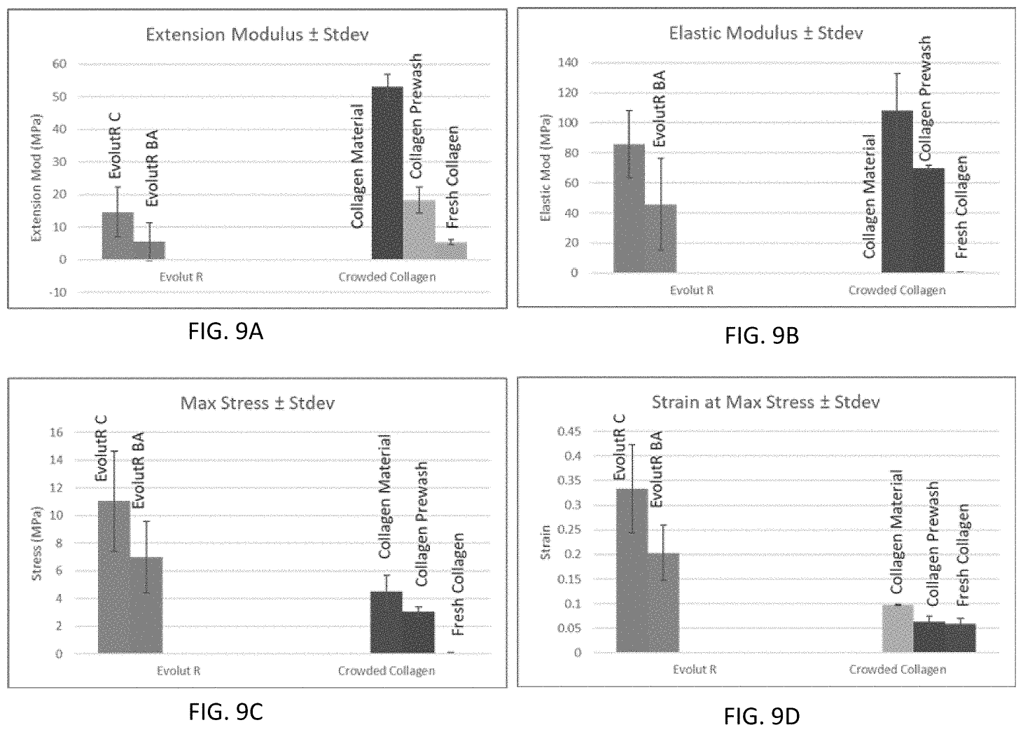

The resulting tissue was also tested for its ability to resist enzymatic (pronase) degradation. Resistance is defined as the pre-digestion weight divided by the post-digestion weight. The collagen material, bovine collagen tissue, and porcine collagen tissue were subjected to pronase degradation. The resistance to pronase degradation is shown in FIG. 10, which shows that the collagen material made according to the teachings presented herein is at least as resistant, if not more resistant, to pronase degradation as porcine and bovine collagen tissue.

Higher concentration collagen material (about 250 mg/ml and about 700 mg/ml) was also prepared. The material had higher elastic modulus and max stress values (data not shown).

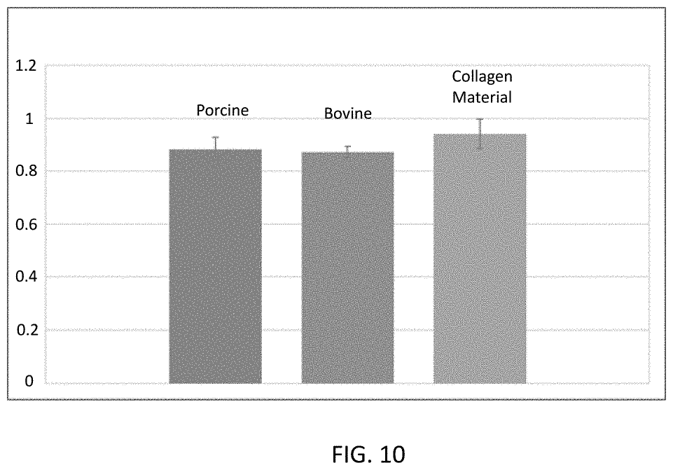

The resulting collagen materials were visually evaluated via microscopy. FIGS. 11A-D shows the 150 mg/ml collagen material (before and after rinsing) at 6000× magnification, the 250 mg/ml collagen material (after rinsing) at 6000× magnification, and Evolut™ Pro tissue at 3000×. As can be seen, the 250 mg/ml collagen material is denser than the 150 mg/ml collagen material and they both include a network of branched fibrils.

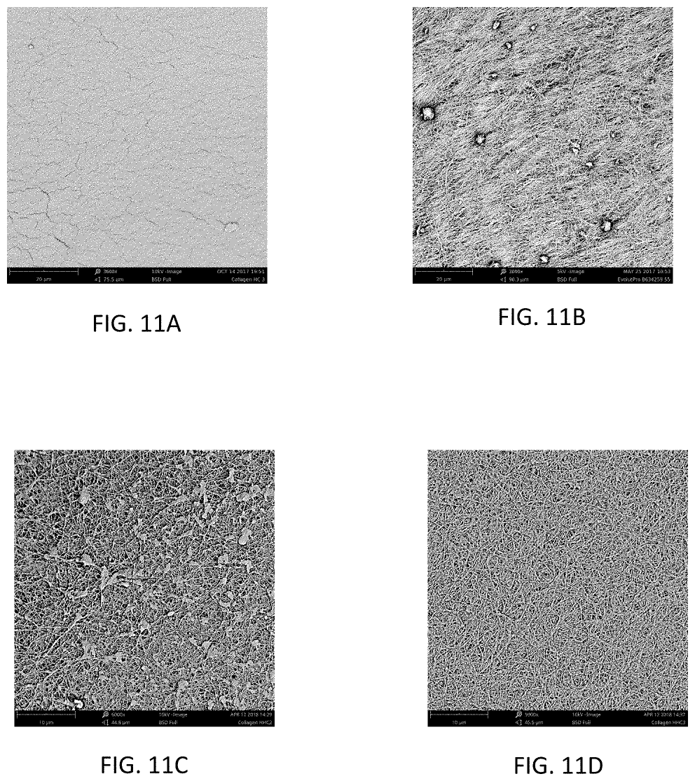

Collagen material was made substantially as described above, but with fours hours incubation at 4° C. after neutralization, followed by incubation at 37° C. for 1 hour. FIGS. 12A-B shows 6000× magnification of non-rinsed (left) and rinsed (right) images of the resulting collagen material. As shown in the right panel, larger diameter fibers are evident denoting capability of this method to produce collagen fibers.

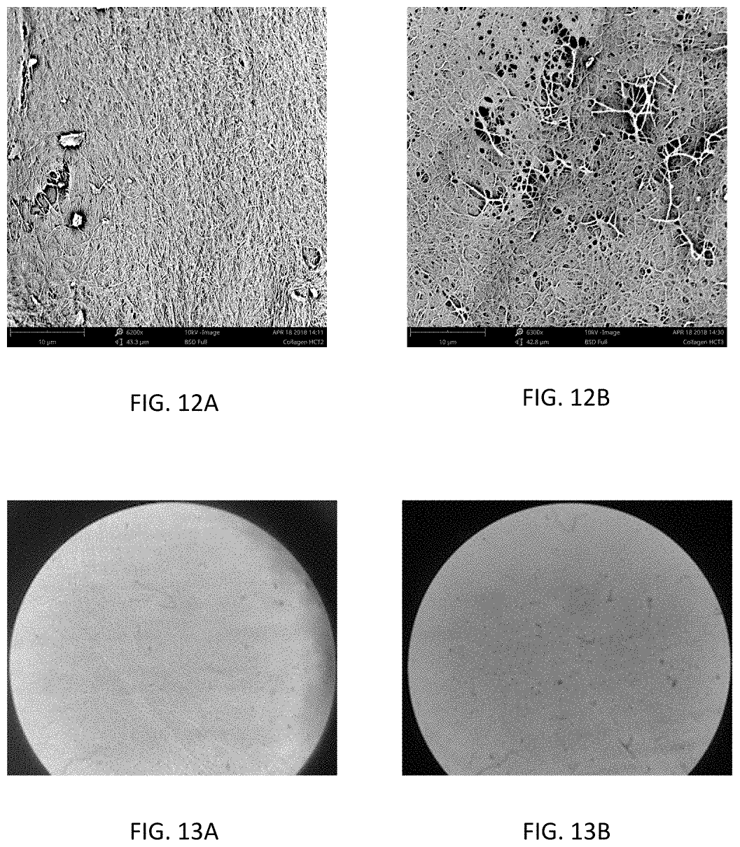

Collagen material was formed in dialysis cassettes with and without bidirectional flow of 300 L/hour. In case of bidirectional flow, two submersible pumps were used to generate flows on each surface of the sheets that where the flow directions were perpendicular to each other. Images of resulting collagen material is show in FIGS. 13A-B. As shown in the right panel (bidirectional flow) vs. the left panel (no flow), enhanced fiber orientation and increased fiber thickness may be achieved with flow. Results of uniaxial testing are shown below in Table 1. As indicated by FIGS. 13A-B and Table 1, the properties of the collagen material may be modified by adjusting the process for forming the collagen material.

TABLE 1
Mechanical Properties of Collagen Material Subjected to Flow During
Crowding

| | | Elastic | | | |
|---|---|---|---|---|---|
| | | Modulus | Max Stress or | Strain at Max | Thickness |
| Flow | n | (MPa) | UTS (MPa) | Stress | (mm) |
| Yes | 3 | 65.8 ± 1.8 | 5.7 ± 0.6 | 0.14 ± 0.002 | 0.157 ± 0.02 |
| No | 3 | 108.1 ± 22.3 | 4.5 ± 1.2 | 0.06 ± 0.01 | 0.174 ± 0.03 |

Collagen material can include varying degree of fibronectin or laminin or periostin or any other native heart valve protein or a combination thereof in its structure. As an example, 40 ug/ml fibronectin was added to the collagen material solution during neutralization stage. The same material underwent overcrowding during dialysis by selecting 10K MWCO for membrane and 8K MW for PEG. The resulting sheets showed enhanced mechanical properties as shown below in Table 2. The amount of fibronectin added may be at a concentration of 1 ug/ml or more, such as 10 ug/ml or more, 40 ug/ml or more, or 100 ug/ml or more.

TABLE 2
Mechanical Properties of Collagen Material with fibronectin and
overcrowding

| | | Max | | |
|---|---|---|---|---|
| | Elastic Modulus | Stress or | Strain at Max | |
| n | (MPa) | UTS (MPa) | Stress | Thickness (mm) |
| 3 | 78.02 | 7.65 | 0.14 | 0.191 |

Collagen material was formed and subjected to crosslinking with various different cross-linking agents at various concentrations for various amounts of time. The cross-linking agents tested included glutaraldehyde, hexamethylene diisocyanate (HMDI), and genipin. The various agents and conditions affected the resulting mechanical properties and shrinkage temperatures of the resulting collagen materials (data not shown).

In the foregoing detailed description, the invention has been described with reference to specific embodiments. However, it may be appreciated that various modifications and changes can be made without departing from the scope of the invention as set forth in the appended claims. Furthermore, various combinations of elements described above in conjunction with the specific embodiments, are within the scope of the present invention, for example, according to the appended claims.

1. A method comprising:
forming collagen fibrils; and
crowding or over crowding the collagen fibrils to form a collagen construct having a collagen density of 100 mg/ml or greater.

2. The method of claim 1, wherein the collagen construct has a collagen density of 250 mg/ml or greater.

3. The method of claim 1, wherein the collagen construct has a collagen density from 700 mg/ml to 1000 mg/ml.

4. The method of claim 1, wherein the collagen fibrils are concentrated and crowded or overcrowded via dialysis.

5. The method of claim 4, wherein the collagen fibrils are concentrated by dialysis using a membrane having a molecular weight cut off in a range from about 1000 to about 20,000.

6. The method of claim 4, wherein the collagen fibrils are crowded or overcrowded by dialysis against a solution comprising polyethylene glycol (PEG).

7. The method of claim 4, wherein the PEG has a number average molecular weight (Mn) in a range from about 2000 to about 30,000.

8. The method of claim 1, further comprising providing an acidic solution comprising collagen monomers and neutralizing the acidic solution to a pH from about 5 to about 10 to form the collagen fibrils.

9. The method of claim 8, wherein the concentration of collagen monomers in the solution is about 50 mg/ml or less.

10. The method of claim 8, further comprising heating the neutralized solution that had been maintained at about 4° C. to a temperature of about 37° C. for about 15 mins to about 4 hours.

11. The method of claim 1, further comprising pre-incubating the neutralized solution at about 4° C. for about 4 hours to 48 hours.

12. The method of claim 1, further comprising applying a load to the collagen fibrils during the crowding of the collagen fibrils.

13. The method of claim 1, wherein crowding the collagen fibrils comprises crowding the fibrils with an additional component to form a hybrid structure, wherein the addition component is selected from the group consisting of a fabric, a polymer, a biologic material, and a metal.

14. The method of claim 13, wherein the additional component comprises elastin, fibronectin, laminin, or periostin.

15. An engineered tissue comprising a collagen construct having plurality of intertwined individual collagen fibrils, a network of branched collagen fibrils, collagen supra-fibrils, or collagen fibers, wherein the collagen construct has a density of 100 mg/ml or greater.

16. The engineered tissue of claim 15, wherein the collagen construct has a density of 250 mg/ml or greater.

17. The engineered tissue of claim 15, wherein the collagen construct comprises cross-linked fibrils, supra-fibrils, or fibers.

18. The engineered tissue of claim 15, wherein the collagen construct is free of cells.

19. A prosthetic heart valve comprising prosthetic valve leaflets, wherein the prosthetic valve leaflets comprise the engineered tissue according to claim 15.

20. A paravalvular wrap comprising the engineered tissue according to claim 15.
